(19) United States
(12) Patent Application Publication
(10) Pub. No.: US 2022/0265689 A1
(43) Pub. Date: Aug. 25, 2022

(54) HIV PRE-EXPOSURE PROPHYLAXIS

(71) Applicant: THE UNITED STATES OF AMERICA ,AS REPRESENTED BY THE SECRETARY, DEPARTMENT OF HEALTH AND HUMAN SERVIC, Bethesda, MD (US)

(72) Inventors: Jose Gerardo Garcia Lerma, Decatur, GA (US); Ivana Mabel Massud, Atlanta, GA (US); Walid M. Heneine, Atlanta, GA (US)

(73) Assignee: THE UNITED STATES OF AMERICA , as represented by the Secretary, Department of Health and Human, Bethesda, MD (US)

(21) Appl. No.: 17/628,170

(22) PCT Filed: Jan. 27, 2020
(86) PCT No.: PCT/US2020/015147

§ 371 Date: Jan. 18, 2022

Related U.S. Application Data
(60) Provisional application No. 62/876,539, filed on Jul. 19, 2019.

(51) Int. Cl. A61K 31/675; A61K 31/47; A61K 31/513; A61K 31/5377; A61P 31/18

(52) U.S. Cl. CPC A61K 31/675; A61K 31/47; A61K 31/513; A61K 31/5377; A61P 31/18

(57) ABSTRACT

Disclosed is the use of a nucleoside reverse transcriptase inhibitor, a nucleotide reverse transcriptase inhibitor, and an integrase inhibitor prior to an exposure to a potential human immunodeficiency virus (HIV) infection, to protect the subject from the HIV infection. In some embodiments, a prophylactically effective amount of emtricitabine (FTC), a prophylactically effective amount of tenofovir or a tenofovir prodrug, such as tenofovir alafenamide (TAF) or tenofovir disoproxil fumarate (TDF), a prophylactically effective amount of the integrase inhibitor elvitegravir (EVG), and optionally cobicistat (COBI) are used to inhibit or prevent an HIV infection, wherein these agents are administered only prior to the exposure. In specific non-limiting examples, only one dose of the anti-retroviral viral agents is administered to a subject prior to the exposure.

### CROSS REFERENCE TO RELATED APPLICATIONS

This claims the benefit of U.S. Provisional Application No. 62/876,539, filed Jul. 19, 2019, which is incorporated by reference herein.

### STATEMENT OF GOVERNMENT SUPPORT

This invention was made with Government support under project number 1134 by the Centers for Disease Control and Prevention. The United States Government has certain rights in the invention.

### FIELD OF THE DISCLOSURE

This disclosure generally relates to a process for inhibiting a human immunodeficiency virus (HIV) infection, and in particular to a combination of antiviral drugs useful in pre-exposure prophylaxis against the HIV infection.

### BACKGROUND

Despite the fact that significant progress has been made slowing the advancement of the symptoms of acquired immune deficiency syndrome (AIDS) associated with a human immunodeficiency virus (HIV) infection, in the absence of an effective vaccine, HIV continues to spread globally with 37.9 million people living with HIV-1 at the end of 2018, and an estimated 1.7 million people newly infected in 2018. Antiretroviral therapy of HIV-infected persons resulting in durable HIV suppression has been shown to significantly reduce transmissibility of HIV. However, this approach cannot provide protection from HIV-infected persons who are undiagnosed, untreated, or virologically unsuppressed. Antiretroviral prophylaxis has been advanced as a strategy to prevent HIV acquisition among persons at high risk of HIV infection.

Post-exposure prophylaxis (PEP) methods are often used for the prevention of an HIV infection, but they generally require the administration of anti-retroviral drug combinations for a prolonged period of time (usually 28 days). Although these PEP protocols have reduced the incidence of seroconversion following exposure to HIV, they require clinicians for administration and management for each potential HIV exposure adding cost and complexity. In addition, many patients experience significant adverse drug reactions during prolonged periods of drug administration or are otherwise unable to continue the treatment for the full 28 day period.

Daily oral pre-exposure prophylaxis (PrEP) with the combination of emtricitabine (FTC) and tenofovir disoproxil fumarate (TDF) (the combination is sold as TRUVADA®) is recommended by CDC and WHO for the prevention of HIV in persons at high risk of infection. However, many people find it challenging to adhere to a daily dosing schedule and cannot fully benefit from PrEP. A need remains for an HIV prevention regimen that does not require daily dosing and can prevent infection if taken on-demand (such as before or after high-risk sexual activity).

### SUMMARY OF THE DISCLOSURE

The highly effective pre-exposure prophylaxis methods disclosed herein can avoid the clinical drawbacks of daily doses of anti-retroviral agents and are cost effective. In some embodiments, only one dose is required.

Methods are disclosed herein for protecting a primate from a self-replicating infection by an immunodeficiency virus without the necessity of prolonged pre-exposure administration of an anti-retroviral agent, and without the necessity of post-exposure administration of anti-retroviral agents. Specifically, it is disclosed that co-administration of a nucleoside reverse transcriptase inhibitor, a nucleotide reverse transcriptase inhibitor, and an integrase inhibitor before an exposure of a subject to a potential immunodeficiency virus infection can inhibit or prevent an immunodeficiency virus infection, such as an HIV infection. In further embodiments a pharmacoenhancer can also be co-administered to the subject.

Disclosed are methods for pre-exposure prophylaxis that are of use to prevent or inhibit an immunodeficiency virus infection in a subject. In some embodiments, a prophylactically effective amount of a tenofovir prodrug, such as tenofovir alafenamide (TAF), TDF, or a tenofovir salt, a prophylactically effective amount of elvitegravir (EVG) a prophylactically effective amount of emtricitabine (FTC), and optionally a prophylactically effective amount of cobistat (COBI) are co-administered to inhibit or prevent an HIV infection in a subject, before an exposure of the subject to a potential HIV infection. In specific non-limiting examples, only one or two doses of the anti-retroviral agents are co-administered to a subject before an exposure to a potential HIV infection. In some embodiments these anti-retroviral agents are not administered to the subject after the exposure to the potential immunodeficiency retrovirus infection, such as HIV. In a specific, non-limiting example, only one dose is co-administered to the subject.

The foregoing and other objects, features, and advantages of the invention will become more apparent from the following detailed description, which proceeds with reference to the accompanying figures.

### BRIEF DESCRIPTION OF THE FIGURES

FIGS. 1A-1B. Efficacy of a single oral dose of TAF/FTC/EVG/COBI against simian HIV infection (SHIV). 1A) Study design. 1B) Macaques received TAF/FTC/EVG/COBI orally by gavage at the indicated time points relative to the time of rectal SHIV exposure. Single-dose TAF/FTC/EVG/COBI protected 5/6 macaques in modality 1 and 4/6 macaques in modality 2. In contrast, 7/8 untreated controls were infected. The calculated efficacy of modality 1 and modality 2 in preventing infection was high and significant (91.7% [95% CI=35.7%, 98.9%] and 80.0% [95% CI=10.8%, 95.5%], respectively).

FIGS. 2A-2C. Concentration of SHIV RNA in plasma over time. 2A) Five of the 6 animals in modality 1 (PrEP-4 h) remained uninfected. 2B) Four of the 6 animals in modality 2 (PrEP-24 h) remained uninfected. 2C) In contrast, only one of the 8 untreated controls remained uninfected.

### DETAILED DESCRIPTION OF SEVERAL EMBODIMENTS

Methods are disclosed herein for protecting a subject from a self-replicating infection by an immunodeficiency virus, specifically HIV. These methods include co-administering to the primate a prophylactically effective amount of a nucleoside reverse transcriptase inhibitor, a nucleotide reverse transcriptase inhibitor, and an integrase inhibitor before exposure to a potential immunodeficiency virus infection. In further embodiments, the methods also include co-administering a pharmacoenhancer. In some embodiments, these agents are administered only before an exposure to a potential immunodeficiency retrovirus infection, such as HIV. Thus, in these embodiments these agents are not administered to the subject after the exposure. In specific non-limiting examples, the nucleoside reverse transcriptase inhibitor comprises FTC, the nucleotide reverse transcriptase inhibitor comprises tenofovir or a tenofovir prodrug such as TAF or TDF, and the integrase inhibitor comprises EVG. In other non-limiting examples, the pharmacoenhancer comprises COBI.

### I. Abbreviations

COBI: cobicistat
EVG: elvitegravir
FTC: 2′-deoxy-5-fluoro-3′thiacytidine, also known as emtricitabine
H: hours
HAART: highly active anti-retroviral therapy
HIV: human immunodeficiency virus
TAF: tenofovir alafenamide
TDF: tenofovir disoproxil fumarate
SIV: simian immunodeficiency virus
SHIV: simian/human immunodeficiency virus, a quimeric virus that contains the env coding region
of HIV-1 in in a background of SIV
NARTI or NRTI: Nucleoside analog reverse-transcriptase
NtARTI or NtRTI: Nucleotide analog reverse-transcriptase inhibitors
PEP: Post-exposure prophylaxis
PrEP: Pre-exposure prophylaxis

### BRIEF SUMMARY OF TERMS

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “an agent” includes single or plural agents and can be considered equivalent to the phrase “at least one agent.”

As used herein, the term “comprises” means “includes.” Thus, “comprising an antigen” means “including an antigen” without excluding other elements. Furthermore “about” indicates within 5%, unless otherwise indicated in a specific context.

It is further to be understood that any and all base sizes or amino acid sizes, and all molecular weight or molecular mass values, given for nucleic acids or polypeptides are approximate, and are provided for descriptive purposes, unless otherwise indicated.

To facilitate review of the various embodiments, the following explanations of terms are provided:

Administration: The introduction of a composition into a subject by a chosen route. Administration can be local or systemic. For example, if the chosen route is intravenous, the composition is administered by introducing the composition into a vein of the subject. As used herein, the term “co-administer” (or “co-administration”) refers to administration of two or more agents within about 2 hours of each other, for example, as part of a clinical treatment regimen. In other embodiments, “co-administer” refers to administration of two or more agents within 1 hour of each other. In other embodiments, “co-administer” refers to administration of two or more agents within 30 minutes of each other. In other embodiments, “co-administer” refers to administration of two or more agents within 15 minutes of each other. In other embodiments, “co-administer” refers to administration of two or more agents at the same time, either as part of a single formulation or as multiple formulations that are administered by the same or different routes. A single “dose” refers to co-administration of agents at the same time.

Agent: Any substance or any combination of substances that is useful for achieving an end or result; for example, a substance or combination of substances useful for inhibiting or preventing an immunodeficiency virus, such as an HIV, infection in a subject. Agents include proteins, nucleic acid molecules, compounds, small molecules, organic compounds, inorganic compounds, or other molecules of interest. An agent can include a therapeutic agent (such as an anti-retroviral agent), a diagnostic agent or a pharmaceutical agent.

Animal: Living multi-cellular vertebrate organisms, a category that includes, for example, mammals and birds. The term mammal includes both human and non-human mammals. Similarly, the term “subject” includes both human and veterinary subjects. The term “primate” includes human and non-human primates, such as macaques and rhesus monkeys. Thus, a primate includes a monkey, baboon, chimpanzee, gorilla, and a human. Nonhuman primates are appreciated to themselves be susceptible to infection by retroviruses and in particular immunodeficiency viruses and represent well-established animal models as to human response with an appreciation that physiological differences often require different doses in milligrams per kilogram for a nonhuman primate animal model relative to a human.

Anti-retroviral agent: An agent that specifically inhibits a retrovirus from replicating or infecting cells. Non-limiting examples of antiretroviral drugs include fusion inhibitors (e.g., enfuvirtide), entry inhibitors (e.g., maraviroc), nucleoside and nucleotide reverse transcriptase inhibitors (e.g. lamivudine, zidovudine, abacavir, tenofovir, TAF, TDF, FTC), protease inhibitors (e.g., indinavir, ritonavir, darunavir, atazanavir), and integrase inhibitors (e.g., elvitegravir, raltegravir, dolutegravir).

Anti-retroviral therapy (ART): A therapeutic treatment for HIV infection involving administration of at least one anti-retroviral agent (e.g., one, two, three or four anti-retroviral agents) to an HIV-infected individual during a course of treatment. One example of a regimen includes treatment with a combination of TAF, FTC, and EVG. In some examples, ART includes Highly Active Anti-Retroviral Therapy (HAART).

Cobicistat (COBI): 1,3-thiazol-5-ylmethyl (2R,5R)-(5-{[(2S)-2-[(methyl{[2-((propan-2-yl)-1,3-thiazol-4-yl]methyl}carbamoyl)amino]]-4-(morpholin-4-yl)butanamido}-1,6-diphenylhexan-2-yl)carbamate. Cobicistat is a cytochrome P450 3A inhibitor that acts as pharmacokinetic enhancer to increase the effectiveness of HIV anti-retroviral drugs. It is used to increase the bioavailability of other anti-retroviral agents. Cobicistat is marketed as TYBOST® and is also known as GS-9350.

Diurnal: Any pattern that recurs daily.

Effective amount: The amount of an agent (such as an anti-retroviral agent) that alone, or together with one or more additional agents, induces the desired response, such as an inhibition of an HIV infection.

Emtricitabine; 2′-deoxy-5-fluoro-3′thiacytidine (FTC). FTC is sold under the trade name EMTRIVA® (emtricitabine) formerly COVIRACIL®), is a nucleoside reverse transcriptase inhibitor (NRTI) used in the treatment of HIV infection in adults and children. Emtricitabine is also marketed in a fixed-dose combination with tenofovir disproxil fumerate (Viread) under the brand name TRUVADA®. A fixed-dose triple combination of emtricitabine, tenofovir and efavirenz (Sustiva, marketed by Bristol-Myers Squibb) was approved by the U.S. Food and Drug Administration (FDA) on Jul. 12, 2006 under the name ATRIPLA®. Emtricitabine makes up one fourth of the four drug (“Quad”) combination known as STRIBILD®.

Exposure: Contact with an agent, such as a virus, in an environment. An exposure to a potential infection, such as a fungus, virus or bacterium, is an exposure to an environment wherein it is possible that that the infectious agent (the fungus, virus or bacterium, respectively) is present. An exposure can be, for example, an occupational or sexual exposure.

Human Immunodeficiency Virus (HIV): A retrovirus that causes immunosuppression in humans (HIV disease) and leads to a disease complex known as the acquired immunodeficiency syndrome (AIDS). “HIV disease” refers to a well-recognized constellation of signs and symptoms (including the development of opportunistic infections) in persons who are infected by an HIV virus, as determined by antibody or western blot studies. Laboratory findings associated with this disease include a progressive decline in T cells. HIV includes HIV type 1 (HIV-1) and HIV type 2 (HIV-2). Related viruses that are used as animal models include simian immunodeficiency virus (SIV), simian/human immunodeficiency virus (SHIV), and feline immunodeficiency virus (FIV). Treatment of HIV-1 with ART has been effective in reducing the viral burden and ameliorating the effects of HIV-1 infection in infected individuals.

Inhibiting or protecting from a disease: Inhibiting the full development of a disease or condition, for example, in a subject who is at risk for a disease such as acquired immunodeficiency syndrome (AIDS). “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters well known in the art that are specific to the particular disease. A “prophylactic” or “protective” treatment is a treatment administered to a subject who does not exhibit signs of a disease. A “protective” treatment for an immunodeficiency virus inhibits infection of the subject when the subject is subsequently exposed to the virus, or inhibits the HIV infection from developing to a self-replicating infection. A protective treatment results in the subject being serologically negative and negative in a polymerase chain reaction (PCR) testing for viral genome.

Nucleoside analog reverse-transcriptase inhibitors (NRTIs): The initial class of antiretroviral drugs that was developed. In order to be incorporated into the viral DNA, NRTIs must be activated in the cell by the addition of phosphate groups to their deoxyribose moiety, to form NRTI triphosphates. This phosphorylation step is carried out by cellular kinase enzymes. NRTIs include zidovudine, didanosine, zalcitabine, stavudine, lamivudine, abacavir, and emtricitabine (also called FTC).

Nucleotide analog reverse-transcriptase inhibitors (NtRTIs): NTARTIs and NtRTIs are nucleotide analogues of cytidine, guanosine, thymidine, and adenosine that are of use in treatment of HIV infections. For example, tenofovir is an NtRTI adenosine analogue.

Pharmacoenhancer: A substance that increases the bioavailability and bioefficacy of active substances with which they are combined without having any activity of their own at the dose used. These agents are also known as “bioenhancers.” Increased bioavailability means increased levels of an agent, such as in the blood. Increased bioefficacy means the increased effectiveness of the drug due to, at least in part, to increased bioavailability. COBI is a pharmacoenhancer.

Tenofovir Prodrugs: Tenofovir (9-R-[(2-phosphonomethoxy)propyl]adenine), an acyclic nucleotide analog of dAMP, is a potent in vitro and in vivo inhibitor of human immunodeficiency virus type 1 (HIV-1) replication. Tenofovir is sequentially phosphorylated in the cell by AMP kinase and nucleoside diphosphate kinase to the active species, tenofovir diphosphate, which acts as a competitive inhibitor of HIV-1 reverse transcriptase that terminates the growing viral DNA chain. Tenofovir disoproxil fumarate (TDF) is an oral prodrug of tenofovir, marketed as VIREAD®, that has received marketing authorization in many countries as a once-daily tablet (300 mg) in combination with other antiretroviral agents for the treatment of HIV-1 infection.

U.S. Pat. Nos. 7,390,791 and 7,803,788, both incorporated herein by reference, disclose prodrugs of phosphonate nucleotide analogs that are also useful in therapy. [(R)-2-[[(S)-[[(S)-1-(isopropoxycarbonyl)ethyl]amino] phenoxyphosphinyl]-methoxy]propyl]adenine 16 is an isopropylalaninyl phenyl ester prodrug of tenofovir. Tenofovir alafenamide (TAF) is also known as GS-7340. TAF has been marketed under the name VEMLIDY®. The hemifumarate form of TAF is also of use in the methods disclosed herein. TAF exhibits potent anti-HIV activity 500- to 10300-fold enhanced activity relative to tenofovir against HIV-1 T cells, activated peripheral blood mononuclear lymphocytes (PBMCs), and macrophages. TAF also has enhanced ability to deliver and increase the accumulation of the parent tenofovir into PBMCs and other lymphatic tissues in vivo. TAF can be prepared as described in U.S. Pat. No. 7,390,791, incorporated herein by reference.

FTC/TAF/EVG/COBI, also called GENVOYA® (which contains 150 mg EVG, 150 mg COBI, 200 mg FTC, and 10 mg TAF) is approved for the treatment of an existing HIV infections in subjects. EVG, FTC and TAF have been shown to suppress viral reproduction. Cobicistat increases the effectiveness of the combination, such as by inhibiting the liver and gut wall enzymes that metabolize EVG. The use of FTC/TAF/EVG/COBI for treatment of an existing HIV infection is disclosed, for example, in U.S. Patent Publication U.S. 2015/0105350 entitled “Combination Therapy Comprising Tenofovir Alafenamide Hemifumarate and Cobicistat for Use in the Treatment of Viral Infections,” which is incorporated herein by reference. A FTC/TAF/EVG/COBI combination drug is manufactured, and is commercially available from, Gilead Sciences.

Therapeutic agent: Used in a generic sense, it includes treating agents, prophylactic agents, and replacement agents. A therapeutic agent is used to ameliorate a specific set of conditions in a subject with a disease or a disorder.

Therapeutically effective amount and prophylactically effective amount: A quantity of a specific substance, such as a disclosed agent, sufficient to achieve a desired effect in a subject being treated. A therapeutically effective amount can be the amount necessary to inhibit an immunodeficiency virus replication or treat AIDS in a subject with an existing infection with the immunodeficiency virus. “Prophylactically effective amounts” refers to administration of an agent (or combination) that inhibits or prevents establishment of a self-replicating infection with an infectious agent, such as an immunodeficiency virus, for example the Human Immunodeficiency Virus (HIV). Post-exposure prophylaxis (PEP) is the prevention or inhibition of an immunodeficiency virus infection, wherein the active agent(s) are administered after a exposure to an immunodeficiency virus such as HIV. The exposure can be recreational (sexual, drug related, etc.) or occupational (such as from a needle stick or contaminated blood product in the hospital setting). Pre-exposure prophylaxis (PrEP) is the prevention or inhibition of an immunodeficiency virus infection in a host, wherein the active agent(s) are administered prior to any possible infection (e.g., prior to any exposure) of the subject with the virus. As noted above, a “protective” treatment for an immunodeficiency virus inhibits infection of the subject when the subject is subsequently exposed to the virus, or inhibits the HIV infection from developing to a self-replicating infection. “Protection” as used in the context of a host response to an immunodeficiency virus challenge results in the host being serologically negative and negative in a polymerase chain reaction (PCR) testing for viral genome, such as for HIV.

Unit dosage form: A physically discrete unit, such as a capsule, tablet, or solution, that is suitable as a unitary dosage for a human patient, each unit containing a predetermined quantity of one or more active ingredient(s) calculated to produce a therapeutic effect, in association with at least one pharmaceutically acceptable diluent or carrier, or combination thereof. Unit dosage formulations contain a daily dose or an appropriate fraction thereof, of the active ingredient(s).

Virus: Microscopic infectious organism that reproduces inside living cells. A virus consists essentially of a core of a single nucleic acid surrounded by a protein coat, and has the ability to replicate only inside a living cell. “Viral replication” is the production of additional virus by the occurrence of at least one viral life cycle. A virus may subvert the host cells' normal functions, causing the cell to behave in a manner determined by the virus. For example, a viral infection may result in a cell producing a cytokine, or responding to a cytokine, when the uninfected cell does not normally do so.

“Retroviruses” are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-I and HIV-II), simian immunodeficiency virus (SIV), and feline immunodeficiency virus (FIV).

Suitable methods and materials for the practice or testing of this disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and supplements to 2012); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In case of conflict, the present specification, including explanations of terms, will control.

### II. Description of Several Embodiments

The use of a combination of antiretroviral agents as a PrEP treatment is provided herein for protecting a subject from an infection, such as inhibiting an human immunodeficiency virus infection developing to a level of self-replicating infection. Retroviral transmission through most routes entails a new primate host receiving a small number of viral particles. The exposure can be an occupational or recreational exposure. Common routes of retrovirus transmission illustratively include sexual intercourse, medical worker skin puncture inoculation, hypodermic needle sharing, blood transfusions, inadvertent exposure to infected blood products, birth canal exposure, and breastfeeding. In some embodiments, the exposure is through sexual contact. The disclosed methods provide a PrEP treatment for protecting a primate, such as a human from infection with an immunodeficiency retrovirus, such as HIV-1. In some embodiments, the disclosed methods are used in adults, such as humans 18 years or older, that are at risk for exposure to HIV occupationally or recreationally to a HIV infection.

The disclosed method utilized a combination of an NRTI, an NtRTI and an integrase inhibitor that can be compounded by pharmaceutical composition with conventional pharmaceutically acceptable carriers or diluents. Additionally, pharmaceutically acceptable derivatives and prodrugs of active NRTIs, NtRTIs and integrase inhibitors operative in the present methods include salts such as alkali metal salts; esters such as acetate, butyrate, octinoate, palmitate, chlorobenzoates, benzoates, C1-C6 benzoates, succinates, and mesylate; salts of such esters; and nitrile oxides. It is appreciated that other analogs of pharmaceutically active NRTIs, NtRTIs, or integrase inhibitors that provide within a primate host an active antiviral metabolite residue are also suitable for use. A pharmaceutically acceptable carrier or diluent includes agents that are compatible with other ingredients of a dosage and not injurious to a primate host. The identity and process for compounding a combination of at least one NRTI, at least one NtRTI, and at least one integrase inhibitor, into a dosage form suitable for delivery by a route with administration by oral, rectal, topical, vaginal or parenteral routes of administration are provided in Remington's Science and Practice of Pharmacology, 20th Edition, Chapters 37-47, pages 681-929, where parenteral injection includes subcutaneous, intramuscular, intravenous, and intradermal injection.

Prodrugs are also of use, that are compounds that when administered to a primate host generates an active NRTI, NtRTI or integrase inhibitor as a result of spontaneous reaction under physiological conditions, enzymatic catalysis, metabolic clearance, or combinations thereof. An exemplary NtRTI prodrug currently FDA approved for HAART use is TDF and is detailed in U.S. Pat. No. 5,935,946 and or TAF and succinate salts of tenofovir.

The present methods provide an alternative to conventional retroviral therapy using HAART, in response to self-propagating immunodeficiency virus infection by protecting a primate host against the establishment of self-replicating retroviral infection that provides an indication for such therapy. Through prophylactic prior dosing including at least one NRTI, one NtRTI, and one integrase inhibitor, replication of the comparatively low number of viral particles received by a host primate is prevented. To achieve protection against a primate host developing a retroviral self-replicating infection, one dosage, or two dosages, of the NRTI, the NtRTI and the integrase inhibitor is administered to the primate host prior to exposure to the retrovirus. In some embodiments, the NRTI, NtRTI and integrase inhibitor are administered concurrently, such as in a single formulation. This combination can be co-administered with or without a pharmacoenhancer, such as, but not limited to, COBI. In some embodiments, only one dose of the NRTI, NtRTI and integrase inhibitor are administered to the primate host. In further embodiments, only one dose of the NRTI, NtRTI, integrase inhibitor and pharmacoenhancer are administered to the primate host. In some embodiments, the primate host is a human, and the immunodeficiency virus is HIV.

The disclosed methods provide protection against an immunodeficiency virus self-replicating infection through coadministration, such as simultaneous administration, of even a single dosage administered prior to the retroviral exposure. One or two doses can be co-administered within 24 hours prior to retroviral exposure. In specific, non-limiting examples, one dose is administered within 24 hours prior to the exposure. In other specific, non-limiting examples, one or two doses are administered between about 1 to about 24 hours prior to exposure, about 2 to about 24 hours prior to the exposure, about 3 to about 24 hours prior to the exposure or about 4 to about 24 hours prior to the exposure. In specific non-limiting examples, only one dose is administered between about 1 to about 24 hours prior to exposure, about 2 to about 24 hours prior to the exposure, about 3 to about 24 hours prior to the exposure or about 4 to about 24 hours prior to the exposure. Without being bound by theory, the administration of a single dosage in the hours proceeding a potential retroviral exposure is particularly advantageous in assuring compliance and minimizing side effects associated with a more frequent dosing regimen. It is also of use for sporadic recreational or occupational exposure. In particular embodiments, no anti-retroviral agents are administered after the exposure.

In some embodiments, the disclosed methods include co-administering to a primate, such as a human, a combination of a pharmacologically effective amount of the nucleoside reverse transcriptase inhibitor, such as, but not limited to, FTC, a pharmacologically effective amount of the nucleotide reverse transcriptase inhibitor, such as, but not limited to, tenofovir or a tenofovir prodrug such as, but not limited to, a tenofovir salt such as TDF, TAF, or another salt form of tenofovir, and a pharmacologically effective amount of an integrase inhibitor, such as, but not limited to, EVG. This combination can be co-administered with or without a pharmacoenhancer, such as, but not limited to, COBI. In some embodiments, the co-administration is oral. In more embodiments, the co-administration is simultaneous. Pharmaceutically acceptable salts and related forms of the active agent agents can also be used in the disclosed methods. In specific non-limiting examples, only one or two doses of these agents is/are administered to a subject before an exposure and/or one or more doses of these agents are not administered to the subject after to this exposure. For example, the subject does not take a daily post-exposure dose, as in a PEP regimen, with the same anti-retroviral agents. The subject can be human.

In some embodiments, a prophylactically effective amount of nucleoside reverse transcriptase inhibitor, such as, but not limited to, FTC, a prophylactically effective amount of the nucleotide reverse transcriptase inhibitor, such as, but not limited to, tenofovir or a tenofovir prodrug such as TDF or TAF, and a prophylactically effective amount of the integrase inhibitor, such as EVG, can be formulated in a single composition, such as in a unit dose. Optionally, a prophylactically effective amount of a pharmacoenhancer, such as COBI, is included in this same composition. In some embodiments, the composition can be formulated for oral administration. Thus, these active agents can be combined into a single unit dose and administered to the subject of interest.

The doses of individual active components are administered in prophylactically effective amounts, to create a therapeutic concentration of the active composition at the situs of retrovirus initial founder cell population infection. It is appreciated that establishing an effective concentration at the time of viral exposure and replication for a given active agent in the target cells, includes factors for the agent such as the route of administration, pharmacokinetics, absorption rate based on administration route, effects of food on oral absorption, in vivo distribution, metabolic pathways, elimination route, race, gender, and age of the subject, single dose incident side effects, long term administration side effects, and synergistic effects with co-administered active agents. Information related to these factors considered in dosing are available from the United States Food and Drug Administration (fda.gov/oashi/aids/virals.html). In some embodiments, the dosing according to the present methods utilize as a starting point the maximal recommended tolerated dosing levels for the given active agent combination associated with HAART treatment protocols.

In some embodiments, the dose includes oral co-administration of FTC, TAF/TDF/tenofovir/salt, and EVG. In other embodiments, the dose includes oral co-administration of FTC, TAF/TDF/tenofovir/salt, EVG and COBI. In specific non-limiting examples, TAF is included in the dose(s).

U.S. Published Patent Application No. 2015/0105350, incorporated herein by reference, discloses the use of FTC, TAF and other tenofovir prodrugs, EVG, and COBI for the treatment of HIV infections. As disclosed in this published patent application the oral dose of TAF can be in the range from about 0.0001 to about 100 mg/kg body weight per day, for example, from about 0.01 to about 10 mg/kg body weight per day, from about 0.01 to about 5 mg/kg body weight per day, from about 0.5 to about 50 mg/kg body weight per day, from about 1 to about 30 mg/kg body weight per day, from about 1.5 to about 10 mg/kg body weight per day, or from about 0.05 to about 0.5 mg/kg body weight per day. As a non-limiting example, the daily candidate dose for an adult human of about 70 kg body weight will range from about 0.1 mg to about 1000 mg, or from about 1 mg to about 1000 mg, or from about 5 mg to about 500 mg, or from about 1 mg to about 150 mg, or from about 5 mg to about 150 mg, or from about 5 mg to about 100 mg, or about 10 mg, and may take the form of single or multiple doses. In one embodiment, the oral dose of TAF may be in the form of a combination of agents (e.g., TAF/FTC/EVG/COBI).

When COBI or a pharmaceutically acceptable salt thereof is combined with certain specific solid carrier particles (e.g. silica derivatives), the resulting combination possesses improved physical properties. For example, the resulting combination has low hygroscopicity as compared to COBI alone. Additionally, the resulting combination is a free-flowing powder, with high loading values for COBI, acceptable physical and chemical stability, rapid drug release properties, and excellent compressibility. Thus, the resulting combination can readily be processed into solid dosage forms (e.g. tablets). Thus, COBI can be used with any suitable solid carrier, provided the resulting combination has physical properties that allow it to be more easily formulated than the parent compound. For example, suitable solid carriers include kaolin, bentonite, hectorite, colloidal magnesium-aluminum silicate, silicon dioxide, magnesium trisilicate, aluminum hydroxide, magnesium hydroxide, magnesium oxide and talc. In one embodiment, the solid carrier can comprise calcium silicate or magnesium aluminometasilicate. COBI can be coated in the pores and on the surface of a solid carrier. Suitable silica derivatives of use are disclosed in PCT Publication WO 03/037379, incorporated herein by reference.

Exemplary dosages of use in the disclosed methods are (1) COBI: 10-500 mg, 50-500 mg, 75-300 mg, 100-200 mg, or 150 mg; (2) TAF: 1-60 mg, 3-40 mg, 5-30 mg, 8-20 mg, or 10 mg; (3) FTC: 10-500 mg, 50-500 mg, 75-300 mg, 150-250 mg, or 200 mg; and (4) EVG: 10-500 mg, 50-500 mg, 75-300 mg, 100-200 mg, or 150 mg. Tenofovir can be used in amounts of less than 300 mg, 200 mg or less and 100 mg or less. COBI can be used in amounts of 50-500 mg, 100-400 mg, 100-300 mg, and 150 mg.

Tenofovir (or TDF or TAF or another salt form) and COBI or pharmaceutically acceptable salt(s) thereof, can be co-administered. Tenofovir (or TDF or TAF or another salt form), COBI, FTC, and EVG can be co-administered. Tenofovir (or TDF or TAF) and COBI can be co-administered in a single pharmaceutical composition. Tenofovir (or TDF or TAF or another salt form), COBI, FTC, and EVG can be co-administered in a single pharmaceutical composition. One of skill in the art will know that, in the case of administering a pharmaceutically acceptable salt or complex of an agent, the amount administered will be adjusted relative to the weight of the component added to produce the salt or complex.

The method can include co-administering 200 mg of FTC and 150 mg of EVG. The method can include co-administering 150 mg COBI, 100 mg or less tenofovir, 150 mg EVG, and 200 mg FTC. The method can include co-administering 150 mg COBI, 200 mg or less tenofovir, 150 mg EVG, and 200 mg FTC. The method can include co-administering 150 mg COBI, less than 300 mg tenofovir, 150 mg EVG, and 200 mg FTC. The method can include co-administering 150 mg COBI, 50 mg tenofovir, 150 mg EVG, and 200 mg FTC. In some specific non-limiting example, the method can include co-administering 150 mg EVG, 150 mg COB, 200 mg FTC, and 10 mg TAF. These compositions can be administered orally. See U.S. Published Patent Application No. 2015/0105350, incorporated herein by reference for additional dosing information. In some embodiments, GENVOYA® is administered to the primate.

The active agents may be administered to a primate, such as a human, in any conventional manner. While it is possible for the active agents to be administered as raw compounds, they are preferably administered as one or more pharmaceutical compositions. The salt, carrier, or diluent should be acceptable in the sense of being compatible with the other ingredients and not deleterious to the recipient thereof. Examples of carriers or diluents for oral administration include cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), and sodium lauryl sulfate. The pharmaceutical compositions can be prepared by any suitable method, such as those methods well known in the art of pharmacy, for example, methods such as those described in Gennaro et al., Remington's Pharmaceutical Sciences (18th ed., Mack Publishing Co., 1990), especially Part 8: Pharmaceutical Preparations and their Manufacture.

Such methods include the step of bringing into association the active agents with the carrier or diluent and optionally one or more accessory ingredients. Such accessory ingredients include those conventional in the art, such as, fillers, binders, excipients, disintegrants, lubricants, colorants, flavoring agents, sweeteners, preservatives (e.g., antimicrobial preservatives), suspending agents, thickening agents, emulsifying agents, and/or wetting agents. The pharmaceutical compositions of use in the methods disclosed herein can provide controlled, slow release or sustained release of the active agents over a period of time. The controlled, slow release or sustained release of the agents can maintain the agents in the bloodstream of the human for a longer period of time than with conventional formulations.

Pharmaceutical compositions include, but are not limited to, coated tablets, pellets, solutions, powders, capsules, and dispersions in a medium that is insoluble in physiologic fluids, or where the release of the therapeutic compound follows degradation of the pharmaceutical composition due to mechanical, chemical, or enzymatic activity. For oral administration, fine powders or granules may contain diluting, dispersing, and or surface-active agents and may be present, for example, in water or in a syrup, in capsules or sachets in the dry state, or in a non-aqueous solution or suspension wherein suspending agents may be included, or in tablets wherein binders and lubricants may be included. When administered in the form of a liquid solution or suspension, the formulation may contain one or more active ingredients and purified water. Optional components in the liquid solution or suspension include suitable sweeteners, flavoring agents, preservatives (e.g., antimicrobial preservatives), buffering agents, solvents, and mixtures thereof. A component of the formulation may serve more than one function. For example, a suitable buffering agent also may act as a flavoring agent as well as a sweetener. Suitable sweeteners include, for example, saccharin sodium, sucrose, and mannitol. A mixture of two or more sweeteners may be used. The sweetener or mixtures thereof are typically present in an amount of from about 0.001% to about 70% by weight of the total composition. Suitable flavoring agents may be present in the pharmaceutical composition to provide a flavor to make the pharmaceutical composition easier for a human to ingest. The flavoring agent or mixtures thereof are typically present in an amount of about 0.0001% to about 5% by weight of the total composition.

Preservatives can also be present in the composition. Suitable preservatives include, for example, methylparaben, propylparaben, sodium benzoate, and benzalkonium chloride. A mixture of two or more preservatives may be used. The preservative or mixtures thereof are typically present in an amount of about 0.0001% to about 2% by weight of the total composition. Buffering agents can also be present in the compositions. Suitable buffering agents include, for example, citric acid, sodium citrate, phosphoric acid, potassium phosphate, and various other acids and salts. A mixture of two or more buffering agents may be used. The buffering agent can be present in an amount of about 0.001% to about 4% by weight of the total composition.

A solvent can be used when a liquid suspension is desirable. Suitable solvents for a liquid solution or suspension include, for example, sorbitol, glycerin, propylene glycol, and water. A mixture of two or more solvents may be used. The solvent or solvent system can present in an amount of about 1% to about 90% by weight of the total composition. These types of formulations are disclosed, for example, in U.S. Published Patent Application No. 2015/0105350, which is incorporated herein by reference.

In some embodiments, only a limited number of doses of the therapeutic agents are co-administered to a primate before an exposure to the immunodeficiency virus infection. In specific non-limiting examples, only 1, 2, 3, 4 or 5 doses of the therapeutic agents are co-administered to the primate before an exposure to the immunodeficiency virus infection. In further non-limiting examples, only one or two doses of the therapeutic agents are co-administered to the primate before an exposure to the immunodeficiency virus. In another non-limiting example, only one dose of the therapeutic agents is co-administered to the primate before an exposure to the immunodeficiency virus. In further aspects of these embodiments, doses of the therapeutic agents are not administered to the subject after the exposure to the immunodeficiency virus. The primate can be a human subject, and the immunodeficiency virus can be HIV-1.

In additional embodiments, doses of the active agents, e.g., TAF/TDF/tenofovir/salts, FTC, EVG and optionally COBI, are co-administered to the primate only before an exposure to a potential immunodeficiency virus infection. In some non-limiting examples, doses of these agent are not co-administered to the primate after the exposure. In yet other embodiments, these agents are not co-administered to the subject diurnally after to the exposure. Thus, in some embodiments, the primate is not co-administered TAF/TDF/tenofovir/salt, FTC, EVG, and optionally COBI, such as diurnally, after to the exposure. In further non-limiting examples, the primate is not co-administered TAF/TDF, FTC, EVG, and optionally COBI, such as diurnally, for at least 30, 35, 40, 45, 50, 55, 60, 90, or 120 days after the exposure.

In further embodiments, active agents (such as TAF/TDF/tenofovir/salt, FTC, EVG and optionally COBI) are co-administered to a subject at specific time points. In some embodiments, the present methods utilize doses of agents. In some embodiments, each dose can include oral co-administration of FTC, TAF/TDF/tenofovir/salt, and EVG. In other embodiments, each dose can include oral co-administration of FTC, TAF/TDF, EVG and COBI. In specific non-limiting examples, TAF is included in the dose(s).

In some embodiments, the methods include oral administration of FTC, TAF/TDF, and EVG. In other embodiments, the method include oral administration of FTC, TAF/TDF, EVG and COBI. In specific non-limiting examples, TAF is utilized.

In some non-limiting examples, only 1, 2, 3, or 4 doses are co-administered to the subject within about 24, 12, or 4 hours prior to an exposure to a potential human immunodeficiency virus infection. In some non-limiting examples, one or two doses are co-administered to the subject within about 24, 12, or 4 hours prior to the exposure. In other non-limiting examples, one dose can be co-administered to the subject within about 24 hours, within about 12 hours, or within about 4 hours, prior to the exposure.

In further non-limiting examples, only one dose is co-administered to the subject within about 24 hours, within about 12 hours, or within about 4 hours before the exposure to the potent immunodeficiency retrovirus infection. In a further non-limiting example, only one does is co-administered to the subject between about 2 and about 24 hours before the exposure. In a yet another non-limiting example, only one does is co-administered to the subject between about 4 and about 24 hours before the exposure. In another non-limiting example, only one dose is co-administered to the subject within about 2 to about 4 hours before the exposure. In further examples, only one dose is co-administered at about 1, 2, 3, 4, 5, 6, 7, 8, 9, 10, 11, 12, 13, 14, 15, 16, 17, 18, 19, 20, 21, 22, 23, or 24 hours before an exposure to a potential immunodeficiency virus infection. In some embodiments, each dose can include oral co-administration of FTC, TAF/TDF/tenovovir/salt, and EVG. In other embodiments, each dose can include oral co-administration of FTC, TAF/TDF, EVG and COBI. In specific non-limiting examples, TAF is included in the dose(s).

In some non-limiting examples, one dose can be co-administered to the subject, within about 24 hours, within about 12 hours, or within about 4 hours, prior to the exposure to the potential immunodeficiency retrovirus infection. In more non-limiting examples, one dose can be co-administered to the subject at about 24 hours, about 12 hours, or about 4 hours, prior to the exposure. In another one non-limiting example, one dose can be co-administered at within about 24 hours prior to the exposure. In another one non-limiting example, one dose can be co-administered at about 24 hours prior to the exposure. In another one non-limiting example, one dose can be co-administered at within about 4 hours prior to the exposure. In another one non-limiting example, one dose can be co-administered at about 4 hours prior to the exposure. In some embodiments, the dose can include oral co-administration of FTC, TAF/TDF/tenofovir/salt, and EVG. In other embodiments, each dose can include oral co-administration of FTC, TAF/TDF/tenofovir/salt, EVG and COBI. In specific non-limiting examples, TAF is included in the dose(s).

In additional non-limiting examples, two doses are co-administered to the subject within about 24 hours before the exposure to the potential immunodeficiency virus infection. In further non-limiting examples, one or two doses are co-administered to the subject within about 24 hours before the exposure, such as at about 4 hours before the exposure and at 24 hours before the exposure. In some embodiments, each dose can include oral co-administration of FTC, TAF/TDF/tenovovir/salt, and EVG. In other embodiments, each dose can include oral co-administration of FTC, TAF/TDF, EVG and COBI. In specific non-limiting examples, TAF is included in the dose(s).

In other non-limiting examples, the method can include co-administering a dose between about 1 and about 12 hours before the exposure to the potential immunodeficiency virus infection. Thus, in some non-limiting examples, the method can include co-administering a dose at 1, 2, 3, 4, 5, 6, 7, 8, 9, 10, 11 or 12 hours before the exposure. In one non-limiting example, the method can include co-administering a dose about 4 hours before the exposure. In some embodiments, each dose can include oral co-administration of FTC, TAF/TDF/tenovovir/salt, and EVG. In other embodiments, each dose can include oral co-administration of FTC, TAF/TDF, EVG and COBI. In specific non-limiting examples, TAF is included in the dose(s).

In some embodiments, a dose(s) is/are not administered diurnally to the subject after to the exposure to the potential immunodeficiency retrovirus infection. Thus, in some embodiments, dose(s) are co-administered to the subject only before the exposure. When active agents in the dose are not co-administered, such as diurnally, to the subject after the exposure, the subject is not administered to active agents in the dose, for example, at least 30, 35, 40, 45, 50, 55, 60, 90, or 120 days after the exposure. In some embodiments, the subject is not administered the active agents in the dose, for example, at least 1, 2, 3, 4, 5, 6, 7, 8, 19 10, 11 or 12 months after to the exposure. In some embodiments, FTC, TAF/TDF/tenovovir/salt, and EVG are not administered after the exposure. In other embodiments, FTC, TAF/TDF, EVG and COBI are not administered after the exposure.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

### Examples

An “on demand” HIV prevention regimen was developed that uses a combination of antiviral drugs that significantly reduce the risk of sexual HIV infection. It is advantageous to have a highly effective but simple “on demand” regimen that can be self-administered by the user exclusively before an exposure to HIV that does not required repeated administration, such as daily administration. It was investigated if a combination containing two HIV reverse transcriptase inhibitors [FTC and tenofovir alafenamide (TAF)] and an HIV integrase inhibitor could prevent infection with simian HIV (SHIV) in macaques. This combination of drugs is novel for HIV prevention as it contains an integrase inhibitor that acts at later stages of the HIV replicative cycle and can extend the window of protective activity. Elvitegravir (EVG) was used as a prototype integrase inhibitor, and the incorporated pharmacoenhancer cobicistat (c) was also utilized to boost EVG concentrations in plasma. TAF/FTC/EVG/COBI was effective for preventing sexual HIV infection as a single self-administered “before-sex” PrEP dose. The high potency of combining two reverse transcriptase and an integrase inhibitor had high efficacy and provided a long window for self-administration.

The efficacy of a single dose of TAF/FTC/EVG/COBI in preventing an immunodeficiency virus infection was investigated using an established macaque model consisting of repeated exposures to 10 tissue culture infectious doses of an R5-tropic SHIV (SHIV162p3) isolate. Rhesus macaques were exposed rectally to SHIV once a week for up to 8 weeks or until an animal became SHIV RNA positive. Macaque drug doses were used that were adjusted by allometric scaling and body weight (30 mg/kg EVG, 30 mg/kg COBI, 20 mg/kg FTC, and 1.5 mg/kg TAF). These drug doses could be reduced or increased. The following modalities were evaluated:
Modality 1 (-4 h): Macaques (n=6) were exposed rectally to SHIV once a week for a total of 8 exposures, and received one single oral dose of TAF/FTC/EVG/COBI orally 4 h before each weekly exposure.
Modality 2 (-24 h): Macaques (n=6) were exposed rectally to SHIV once a week for a total of 8 exposures, and received one single oral dose of TAF/FTC/EVG/COBI orally 24 h before each weekly exposure.
Untreated control group: Macaques (n=8) received saline as placebo.
   Animals were considered protected against infection if they remained seronegative for SHIV antibodies and negative for SHIV RNA during the 8 weekly virus challenges and the following 16 weeks of drug washout. SHIV RNA in plasma was quantified by an RT-PCR assay with a sensitivity of 50 RNA copies/ml. Virus-specific serologic responses were measured using a synthetic peptide enzyme immunoassay assay.

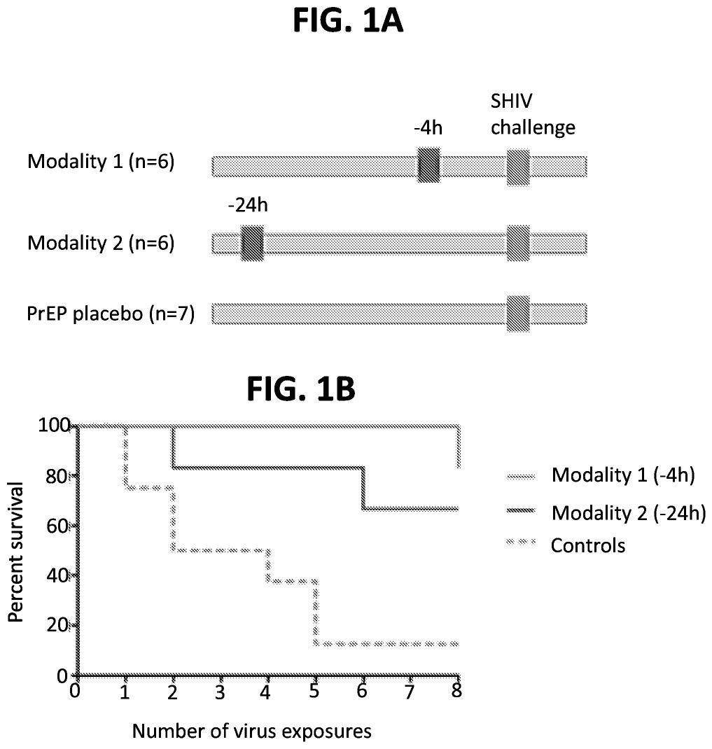

The number of animals protected per group was compared relative to placebo. Modalities 1 and 2 were found to have lower proportion of infection compared to the control group; 1/6 (17%) animals infected in modality 1 (PrEP-4 h) and 2/6 (33%) infected in modality 2 (PrEP-24 h) compared to 7/8 (87.5%) infected in the control group. The two-sided Fisher's exact p values are p=0.026 for modality 1 (PrEP-4 h) and p=0.09 for modality 2 (PrEP-24 h). A survival analysis was also conducted to compare time to infection for the different regimens. The Kaplan-Meier graph in FIG. 1 shows modalities 1 and 2 differed from the untreated control group over time. The two-sided Bonferroni-adjusted p-values are p=0.005 for modality 1 (PrEP-4 h), and p=0.027 for modality 2 (PrEP-24 h). The efficacy of modality 1 and modality 2 in preventing infection was high and significant (91.7% [95% CI=35.7%, 98.9%] and 80.0% [95% CI=10.8%, 95.5%], respectively).

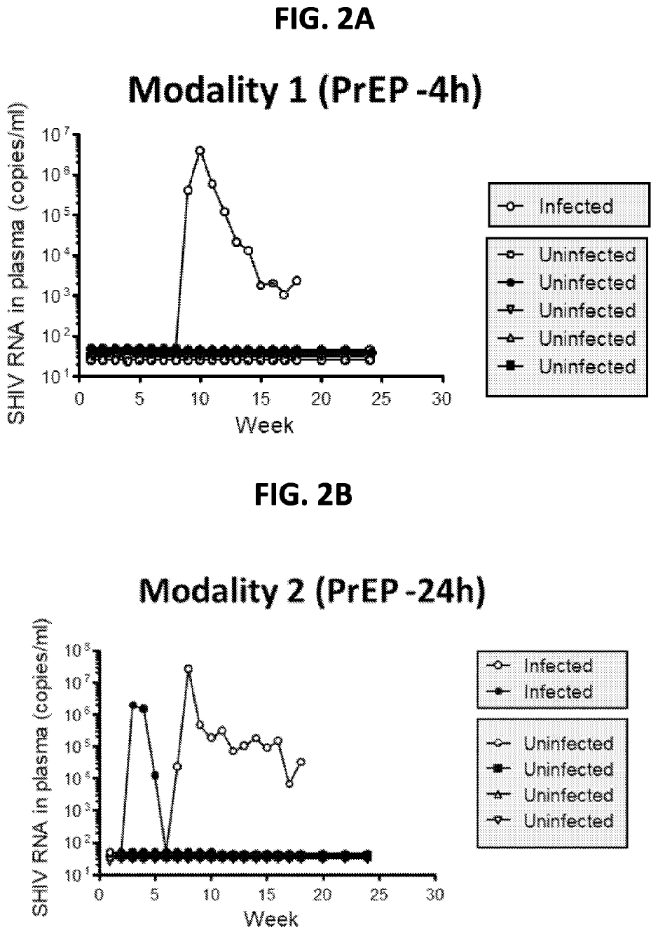

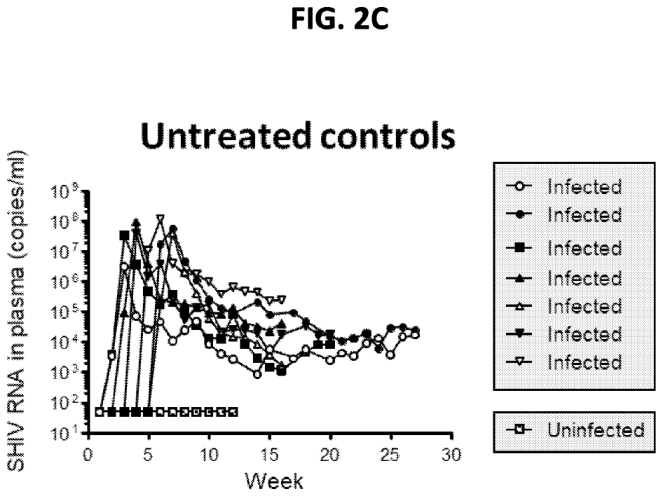

FIG. 2 shows that evidence of systemic SHIV infection as defined by detection of viral RNA in plasma was only observed in 1 out of 6 macaque from modality 1 (PrEP-4 h), and 2 out of 6 macaques from modality 2 (-24 h). In contrast, 7 of the 8 untreated animals were infected with SHIV.

Thus, significant protection was achieved by administering a single time a combination of two HIV reverse transcriptase and one integrase inhibitor before simian HIV exposure, and proof-of-concept of protection against HIV infection was provided by a short and potent self-administered “before sex” PrEP drug combination. This combination can be provided in pill format. The benefits of daily PrEP were surpassed by reducing pill burden and reducing costs. Other integrase inhibitors that are not EVG but that share the same mechanism of action (i.e., blocking integration of HIV in the host genome) can also be used (i.e, raltegravir, dolutegravir, bictegravir, cabotegravir).

1. A method of protecting a subject from a self-replicating infection by a human immunodeficiency virus, comprising co-administering to the subject a prophylactically effective amount of emtricitabine (FTC), a prophylactically effective amount of tenofovir or a tenofovir prodrug, and a prophylactically effective amount of elvitegravir (EVG) prior to an exposure to a potential human immunodeficiency virus infection.

2. The method of claim 1, wherein the tenofovir prodrug is tenofovir alafenamide (TAF) or tenofovir disoproxil fumarate (TDF).

3. The method of claim 2, wherein the tenofovir prodrug is TAF.

4. The method of claim 1, further comprising co-administering a pharmacoenhancer prior to the exposure.

5. The method of claim 4, wherein the pharmacoenhancer comprises cobicistat (COBI).

6. The method of claim 1, wherein FTC, tenofovir or the tenofovir prodrug and the EVG are administered orally to the primate.

7. The method of claim 4, wherein the pharmacoenhancer is administered orally to the primate.

8. The method of claim 5, wherein co-administering comprises a total of 1 or 2 oral doses of FTC, tenofovir or the tenofovir prodrug, EVG and COBI prior to the exposure.

9. The method of claim 8, wherein co-administering comprises a total of 1 oral dose of FTC, tenofovir or the tenofovir prodrug, EVG and COBI prior to the exposure.

10. The method of claim 5, comprising co-administering FTC, tenofovir or the tenofovir prodrug, EVG and COBI only prior to the exposure.

11. The method of claim 8, wherein co-administering comprises co-administering at least one dose of FTC, tenofovir or the tenofovir prodrug, EVG and COBI within about 24 hours prior to the exposure.

12. The method of claim 8, wherein co-administering comprises co-administering at least one dose of FTC, tenofovir or the tenofovir prodrug, EVG and COBI within about 4 hours prior to the exposure.

13. The method of claim 8, wherein co-administering comprises co-administering only one dose of FTC, tenofovir or the tenofovir prodrug, EVG and COBI within about 2 to about 24 hours prior to the exposure.

14. The method of claim 13, wherein co-administering FTC, tenofovir or the tenofovir prodrug, EVG and COBI comprises co-administering only one dose within about 4 to about 24 hours prior to the exposure.

15. The method of claim 14, wherein co-administering FTC, tenofovir or the tenofovir prodrug, EVG and COBI comprises co-administering only one dose within about 4 hours prior to the exposure.

16. The method of claim 14, wherein co-administering FTC, tenofovir or the tenofovir prodrug, EVG and COBI comprises co-administering only one dose at about 4 hours prior to the exposure.

17. The method of claim 14, wherein co-administering FTC, tenofovir or the tenofovir prodrug, EVG and COBI comprises co-administering only one dose within about 24 hours prior to the exposure.

18. The method of claim 14, wherein co-administering FTC, tenofovir or the tenofovir prodrug, EVG and COBI comprises co-administering only one dose at about 24 hours prior to the exposure.

19. The method of claim 1, wherein the FTC, tenofovir or the tenofovir prodrug, and EVG are co-administered in a single pharmaceutical composition.

20. The method of claim 5, wherein the FTC, tenofovir or the tenofovir prodrug, EVG and COBI are co-administered in a single pharmaceutical composition.

21. The method of claim 2, wherein a prophylactically effective amount of the tenofovir prodrug is administered to the subject, and wherein the tenofovir prodrug is TAF.

22. The method of claim 21, wherein the pharmaceutical composition comprises 150 mg elvitegravir, 150 mg cobicistat, 200 mg emtricitabine, and 10 mg tenofovir alafenamide.

23. The method of claim 1, wherein the HIV is HIV-1.

24. A pre-exposure prophylaxis (PrEP) method of protecting a primate subject from a self-replicating infection by an immunodeficiency virus, comprising co-administering to the primate a prophylactically effective amount of a pharmaceutical composition comprising FTC, tenofovir or a tenofovir prodrug, EVG, and COBI prior to exposure to a potential immunodeficiency virus infection, wherein the pharmaceutical composition is not administered after the exposure, and wherein only one or two doses of the pharmaceutical composition is administered to the primate subject.

25. The method of claim 24, wherein the tenofovir prodrug is TAF or TDF.

26. The PrEP method of claim 24, wherein only one dose of the pharmaceutical composition is administered to the primate subject about 4 to about 24 hours prior to the exposure.

27. The PrEP method of claim 24, wherein the only one dose of the pharmaceutical composition is administered within about 24 hours prior to the exposure.

28. The PrEP method of claim 27, wherein the only one dose of the pharmaceutical composition is administered within about 4 hours prior to the exposure.

29. The PrEP method of claim 27, wherein one dose of the pharmaceutical composition is administered at about 24 hours prior to the exposure.

30. The PrEP method of claim 27, wherein one dose of the pharmaceutical composition is administered at about 4 hours prior to the exposure.

31. The PEP method of claim 24, wherein the primate is a human, and wherein the immunodeficiency retrovirus is a human immunodeficiency retrovirus (HIV).

32. The PEP method of claim 31, wherein the HIV is HIV-1.

33. The PEP method of claim 24, wherein a prophylactically effective amount of the tenofovir prodrug is administered to the subject, and wherein the tenofovir prodrug is TAF.

34. The PEP method of claim 33, wherein the pharmaceutical composition comprises 150 mg EVG, 150 mg COBI, 200 mg FTC, and 10 mg TAF.
